(19) United States
(12) Patent Application Publication
(10) Pub. No.: US 2004/0029295 A1
(43) Pub. Date: Feb. 12, 2004

(54) Non-toxic biodegradable microtaggants

(76) Inventors: Brian J. Brogger, Blaine, MN (US); Justin L. Esterberg, Minneapolis, MN (US); David Benderly, New York, NY (US)

Correspondence Address: Beck & Tysver, P.L.L.C., Suite 100, 2900 Thomas Avenue S., Minneapolis, MN, 55416, US

(21) Appl. No.: 10/367,481

(22) Filed: Feb. 14, 2003

Related U.S. Application Data
(60) Provisional application No. 60/356,838, filed on Feb. 14, 2002.

(51) Int. Cl. G01N 33/543

(52) U.S. Cl. 436/523

(57) ABSTRACT

A biodegradable micro particle for tagging food is disclosed. The particle may be of irregular or regular shape and carries optically recognizable indicia on it.

### CROSS REFERENCE TO RELATED CASES

The present application claims the benefit of and incorporates by reference in its entirely U.S. Provisional Application No. 60/356,838 filed Feb. 14, 2002.

### FIELD OF THE INVENTION

The present invention relates generally to the use of micro particles to tag or mark edible products such as foodstuffs. The tags themselves are edible.

### BACKGROUND OF THE INVENTION

Multi-color, layered micro particles called microtaggants have been known to the practice of anti-counterfeiting, authentication, and explosive tracing for decades. These micro particles are typically composed of non-biodegradable synthetic plastics such as melamine polymers. They are unfit for rapid biodegradation with proteases, lipases, or other digestive enzymes. They work well for rapid, on-site visual identification and do not require the use of special tests or other instruments (besides light magnification) like other biodegradable tracers such as those patented by Biotag or Isotag. Single-layered particles with two-dimensional barcodes are also known to us.

### BRIEF DESCRIPTION OF THE DRAWINGS

FIG. 1 is a micro particle in accordance with the invention;

FIG. 2 is a fragmented micro particle in accordance with the invention.

### DETAILED DESCRIPTION

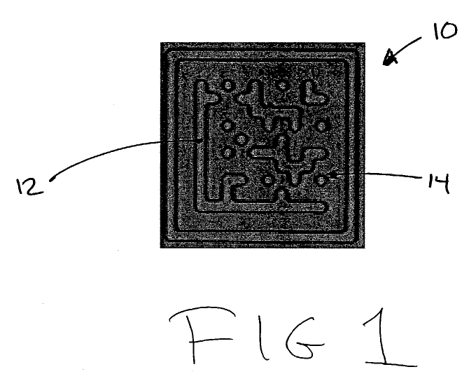

The micro particle of FIG. 1 has a substrate made from a material that is biodegradable and non-toxic the representative particle is about 50 microns on a side. In use, micro particles may be to add to food items that are ingested by humans and by livestock or other animals. The micro particles may also be used in the pharmaceutical industry through their application to pills that are placed in the human digestive tract. Currently there is a problem with counterfeit medications being sold on the black market and there are distribution agreements being broken. The micro particle carries an identifying code that can be used for retrospective identification of the marked product.

The general structure of the particle 10 may be a multilayered with a planform that is roughly a rectangular or square cube with some irregular sides. The shape may also be cylindrical with multi-layered concentric cylinders. Another alternative is a single-layered design with at two-dimensional barcode imprinted, which is illustrated in FIG. 1.

The preferred substrate of the micro particles will be a biodegradable polymer or other substance that is biodegradable. This may include but is not limited to: polysaccharides, starches, polypeptides, proteins, poly-amides, polyglycols, fatty acids, polyester, triglycerides, etc. Specific material compositions include poly-lysine and poly-aspartic acid based polymerrs. Another alternative is to use layers of dead cells or biological tissues, i.e. dried sterile intestinal tissue.

The identification code or indicia can be based on visual colors, fluorescent compounds, phosphorescent compounds, infrared compounds, near-infrared, upconverting phosphors, etc. A possible composition of such micro particles would be to use FDA approved food colorings in multiple layers composed of polysaccharide. Another code system would be to use a two-dimensional barcode system in which the barcode is placed into or onto a particle. The preferred method of making the particle 10 is to stamp or emboss the code onto a planar substrate and to handle the material as a sheet. The sheet may be fragmented thermally to make the micro particles. The embossing technique leaves slight depressions in the particle, which renders them optically readable. Contrast is achieved between the depressed marks and the surrounding material though optical processes that are not well understood at this time. Depressions of just a few microns are sufficient to create sufficient contrast to permit “reading” under the correct illumination. In general the particles may be read in white light using phase contrast microscopy. Other reading methods may be used as well.

The code can be identified by direct visual inspection using magnification under normal white light or other appropriate light conditions such as darkfield fluorescence. The code will be identified prior to inspection (e.g. on the pill prior to administration) or may be read after the animal or human has ingested the material it has been attached to (be it food or drug). In the latter case, the micro particle should not be able to be digested fully so as to make it impossible to read the code. It should be biodegradable post-excretion however.

For other applications it may be desirable to have coded particles that are entirely dissolved in the aqueous solution of the digestive track. Water-soluble polymers may work ideally for this purpose. The particles are therefore edible by humans and are both digested and absorbed or simply excreted.

It is important that the micro particles are non-toxic to animals, humans, and the environment. Generally regarded as safe (GRAS) substances approved by the FDA can be used to satisfy this requirement of being non-toxic to humans and can be incorporated into food inputs and foodstuffs. Many of the aforementioned substances fall into this category and other specific substances listed as GRAS are: polyethylene, polymaleic acid, polysorbates, polyethylene glycol, polypropylene glycol, polyoxypropylene glycol, polyvinylpyrrolidine. Other substances that fall into this category include agar, kelp, rice paper, seaweed paper.

Enclosed are photomicrographs of micro-molded particles, which may be manufactured from rice paper and seaweed. In the FIG. 1 the particle 10 is generally rectangular in shape and is relatively thin. A coordinate system 12 is used to orient robotic vision to determine the location of dots or spots 14 on the particle which are used to create a identifying number or the like. In use the particles will be either cut from or fractured from a laminar substraight with a micro-molded set of taggant zones. This particulate will be mixed with the foodstuff, or adhere to the foodstuff using a conventional adhesive process or a conventional mixing process. In transit and in its final destination the coded particle will retrospectively identify the origin of the foodstuff. Although two-dimensional bar code is a preferred indicia system conventional alphanumeric or other encoding schemes may be used. It is preferred to emboss the code on the particle or mold it in a press however other marking system is within the scope of the invention. The preferred techniques rely on the optical properties of the embossing to provide contrast to read the indicia this in essence requires a 3-D code that has a characteristic depth as well as spatial location on the particle body.

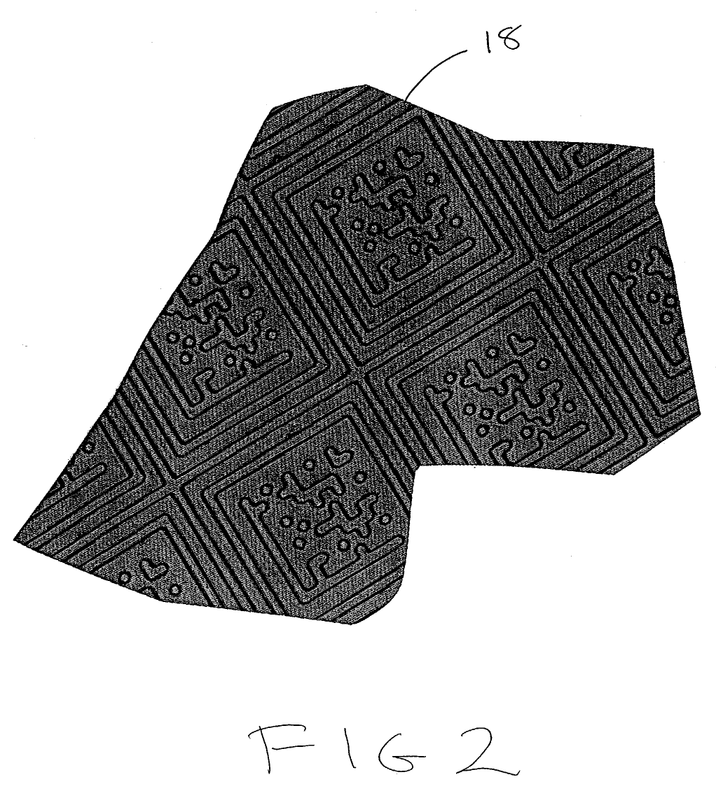

The fracturing technique may take any one of known forms including but not limited to the use of liquid nitrogen to freeze the particle which is then ground producing randomly sized and randomly oriented marked particles. It is important to note that the fracturing process need not produce perfectly rectangular particles the bar code can be reliably read with less than one particle as illustrated by the particle 18 seen in FIG. 2.

What is claimed:

1. A micro particle comprising:
a substantially planar substrate body made from a generally regarded as safe material;
a indicia on said substrate body.

2. The micro particle of claim 1 wherein said indicia is visible in white light.

3. The micro particle of claim 1 wherein said indicia are a two-dimensional bar code based on the spatial location of marks on the substrate body.

4. The micro particle of claim 1 wherein said substrate body is made from a GRAS material.

5. The micro particle of claim 3 where the depth of the mark or height of the mark with respect to the planar substrate encodes information.
